(19) United States
(12) Patent Application Publication
(10) Pub. No.: US 2008/0046054 A1
(43) Pub. Date: Feb. 21, 2008

(54) IMPLANTABLE ELECTRODE ASSEMBLY UTILIZING A BELT MECHANISM FOR SUTURELESS ATTACHMENT

(75) Inventors: Aaron Hjelle, Champlin, MN (US); Mary L. Cole, St. Paul, MN (US); Martin A. Rossing, Coon Rapids, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Maple Grove, MN (US)

(21) Appl. No.: 11/766,592

(22) Filed: Jun. 21, 2007

Related U.S. Application Data
(60) Provisional application No. 60/805,707, filed on Jun. 23, 2006.

(51) Int. Cl. A61N 1/05

(52) U.S. Cl. 607/116; 600/377

(57) ABSTRACT

An electrode assembly for implantation around an elongate biological structure such as, for example, a blood vessel, includes at least one belt mechanism for securing the electrode assembly around the outer surface of the elongate biological structure. Each of the at least one belt mechanism includes a strap and a buckle. The assembly has a length that is sufficient to permit wrapping it around the outer surface of the elongate biological structure. The buckle can be attached to, or integrally formed in, the base, and functions to retain a portion of the strap when the strap is engaged with the buckle.

### CROSS-REFERENCES TO RELATED APPLICATIONS

The present application claims the benefit of provisional U.S. Application No. 60/805,707 (Attorney Docket No. 021433-002300US), filed Jun. 23, 2006, the full disclosure of which is incorporated herein by reference.

The disclosure of this application is also related to U.S. application Ser. No. 11/695,210 (Attorney Docket No.: 021433-002210US), filed Apr. 2, 2007, which claimed the benefit of U.S. Provisional Patent Application No. 60/789,208, entitled “IMPLANTABLE EXTERIOR VESSEL ELECTROSTIMULATION SYSTEM HAVING A RESILIENT CUFF” (Attorney Docket No.: 021433-002200US), filed Apr. 3, 2006, the disclosures of which are incorporated by reference herein in their entirety.

### BACKGROUND OF THE INVENTION

### Field of the Invention

The invention relates generally to medical devices and methods, and more particularly, to an implantable electrode assembly that has features facilitating positioning and securing the assembly during implantation.

Cardiovascular disease is a major contributor to patient illness and mortality. It also is a primary driver of health care expenditure, costing more than $326 billion each year in the United States. Hypertension, or high blood pressure, is a major cardiovascular disorder that is estimated to affect over 50 million people in the United Sates alone. Of those with hypertension, it is reported that fewer than 30% have their blood pressure under control. Hypertension is a leading cause of heart failure and stroke. It is the primary cause of death in over 42,000 patients per year and is listed as a primary or contributing cause of death in over 200,000 patients per year in the U.S. Accordingly, hypertension is a serious health problem demanding significant research and development for the treatment thereof.

Hypertension occurs when the body's smaller blood vessels (arterioles) constrict, causing an increase in blood pressure. Because the blood vessels constrict, the heart must work harder to maintain blood flow at the higher pressures. Although the body may tolerate short periods of increased blood pressure, sustained hypertension may eventually result in damage to multiple body organs, including the kidneys, brain, eyes and other tissues, causing a variety of maladies associated therewith. The elevated blood pressure may also damage the lining of the blood vessels, accelerating the process of atherosclerosis and increasing the likelihood that a blood clot may develop. This could lead to a heart attack and/or stroke. Sustained high blood pressure may eventually result in an enlarged and damaged heart (hypertrophy), which may lead to heart failure.

It has been known for decades that the wall of the carotid sinus, a structure at the bifurcation of the common carotid arteries, contains stretch receptors (baroreceptors) that are sensitive to the blood pressure. These receptors send signals via the carotid sinus nerve to the brain, which in turn regulates the cardiovascular system to maintain normal blood pressure (the baroreflex), in part through activation of the sympathetic nervous system. Electrical stimulation of the carotid sinus nerve (baropacing) has previously been proposed to reduce blood pressure and the workload of the heart in the treatment of high blood pressure and angina. For example, U.S. Pat. No. 6,073,048 to Kieval et al. discloses a baroreflex modulation system and method for stimulating the baroreflex based on various cardiovascular and pulmonary parameters.

Implantable electrode assemblies for electrotherapy or electrostimulation are well-known in the art. For example, various configurations of implantable electrodes are described in U.S. Patent Publication No. U.S. 2004/0010303, which is incorporated herein by reference in its entirety. One type of electrode assembly described therein is a surface-type stimulation electrode that generally includes a set of generally parallel elongate electrodes secured to, or formed on, a common substrate or base typically made of silicone or similar flexible, biocompatible material that is designed to be wrapped around and then typically sutured to the arterial wall. Prior to implantation in a patient, the electrodes are generally electrically isolated from one another. Once the electrode assembly is implanted, one or more of the electrodes are utilized as a cathode(s), while one or more of the remaining electrodes are utilized as an anode(s). The implanted cathode(s) and anode(s) are electrically coupled via the target region of tissue to be treated or stimulated.

The process of implanting the electrode assembly involves positioning the assembly such that the electrodes are properly situated against the arterial wall of the carotid sinus, and securing the electrode assembly to the artery so that the positioning is maintained. The positioning is a critical step because the electrodes must direct as much energy as possible to the baroreceptors for maximum effectiveness and efficiency. The energy source for the implanted baroreflex stimulation device is typically an on-board battery with finite capacity. A high-efficiency implantation will provide a longer battery life and correspondingly longer effective service life between surgeries because less energy will be required to achieve the needed degree of therapy. As such, during implantation of the electrode assembly, the position of the assembly is typically adjusted several times in order to optimize the baroreflex response.

This process of adjusting and re-adjusting the position of the electrode assembly, described as mapping, has been reported by some surgeons as difficult and tedious. Present-day procedures involve positioning and holding the electrode assembly in place with tweezers, hemostat or similar tool while applying the stimulus and observing the response in the patient. Movement by as little as 1 mm can make a difference in the effectiveness of the baroreflex stimulation.

Another challenge related to the mapping process is keeping track of previous desirable positions. Because mapping is an optimization procedure, surgeons will tend to search for better positions until they have exhausted all reasonable alternative positions. Returning the electrode assembly to a previously-observed optimal position can be difficult and frustrating, under surgical conditions.

After determining the optimal position, the surgeon must secure the electrode assembly in place. In the existing technique, this is accomplished by wrapping finger-like elongated portions of the electrode assembly around the artery, applying tension to the material, and suturing the assembly in place. The electrode assembly can be sutured to the arterial wall or to itself (after being wrapped around the artery). Loosening or removing the sutures, re-positioning the electrode assembly, and tightening or re-installing the sutures can increase the time and costs associated with implanting baroreflex activation devices, and can also increase the risk of complications or surgeon errors related to protracted surgical procedures and fatigue.

### BRIEF SUMMARY OF THE INVENTION

According to one aspect of the invention, an electrode assembly is provided for implantation around an elongate biological structure such as, for example, a blood vessel. The electrode assembly includes a generally flexible elastomeric base that has a pair of opposing major surfaces. The base is designed to conform at least partially around an outer surface of the elongate biological structure when the electrode assembly is implanted. A set of electrodes is provided on one of the major surfaces of the base, such as over a bottom surface that is in intimate contact with the elongate biological structure. The electrode assembly includes at least one belt mechanism for selectively securing the electrode assembly around the outer surface of the elongate biological structure.

In one embodiment, each of the at least one belt mechanism includes a strap and a buckle. The strap can be formed integrally with, or attached to, the base, and has a length that is sufficient to permit the electrode assembly to wrap around the outer surface of the elongate biological structure. The buckle can be attached to, or integrally formed in, the base, and functions to retain a portion of the strap when the strap is engaged with the buckle. Optionally, the strap includes surface features, such as protrusions or surface texture, that can increase the friction or adhesive binding force between the strap and the buckle.

In one embodiment, the buckle defines a passage through which the strap can pass. The passage can be a hole, a slit, a tunnel, or the like, and can pass through the base or be situated in parallel along side one of the major surfaces of the base. Optionally, the buckle includes a release tab that can be pulled on, for example, to enlarge or widen the passage.

In one embodiment, the belt mechanism includes a mating set of a through hole and a protrusion that engages with the through hole. The through hole can be defined in the strap and the protrusion can be part of the buckle, or vice-versa.

The belt mechanism according to aspects of the invention can significantly facilitate implantation of the electrode assembly. The belt mechanism can be used together with, or in lieu of, suturing to secure the implanted electrode assembly to the implantation site. The belt mechanism can enable the electrode assembly to be selectively positioned by the surgeon, secured, then loosened, re-positioned, and re-secured. Also, the belt mechanism can facilitate securing the electrode assembly with an appropriate degree of tension.

A method of implanting an electrode assembly around an elongate biological structure according to one aspect of the invention includes providing at least one belt mechanism that includes a strap and a buckle as part of the electrode assembly, wrapping a portion of an outer surface the elongate biological structure with the electrode assembly, and engaging the strap with the buckle such that the buckle retains an engaged portion of the strap. So as to secure the electrode assembly in position around the elongated bridge of the structure.

The step of engaging the strap with the buckle can include threading the at least one strap through a passage defined by the at least one buckle such that the strap and the buckle bind with one another by friction or adhesive retention force. Also, a release tab of the buckle can be pulled on to elastically expand the passage and facilitate the threading, removing, or adjusting of the strap through the passage.

In one embodiment, the engaging of the belt mechanism includes first over-tightening the strap by pulling the strap through the buckle, followed by releasing the strap such that the strap elastically returns towards its un-stretched position at a final tension, and maintaining the final tension. Preferably, the electrode assembly is designed such that the final tension provides a suitable force for securing the electrode assembly to the biological structure.

### BRIEF DESCRIPTION OF THE DRAWINGS

FIG. 1A is a perspective view diagram illustrating an implantable electrode assembly that includes a belt mechanism according to one aspect of the invention.

FIG. 1B illustrates the electrode assembly of FIG. 1A affixed around an arterial wall.

FIG. 2 is a perspective view diagram illustrating a portion of an implantable electrode assembly according to one embodiment in which the straps and buckles are integrally formed with the base.

FIG. 3 is a perspective view diagram illustrating a portion of an implantable electrode assembly according to one embodiment in which the straps are attached to the base.

FIGS. 4A and 4B are each a perspective view diagram illustrating a portion of an implantable electrode assembly according to one embodiment in which the buckles are formed by slits in the base.

FIG. 5A illustrates an electrode assembly according to one aspect of the invention in which the buckles include release tabs.

FIG. 5B illustrates the electrode assembly of FIG. 5A affixed around an arterial wall.

FIGS. 6-8 illustrate various portions of electrode assemblies having examples of different configurations utilizing release tabs on the buckles.

FIGS. 9A-9C illustrate an example electrode assembly having a through hole and mating protrusion type of belt mechanism according to one aspect of the invention.

FIG. 9D illustrates the electrode assembly of FIGS. 9A-9C affixed around an arterial wall.

FIG. 10 illustrates a portion of an implantable electrode assembly according to one embodiment in which the straps have surface features for increasing retention force in the buckle, and in which the base includes apertures useful for marking a position at an implantation site.

FIGS. 11A-1 through 11C-4 illustrate several example embodiments of a belt mechanism strap that includes one or more end features that facilitate threading the strap through the buckle.

### DETAILED DESCRIPTION OF THE INVENTION

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

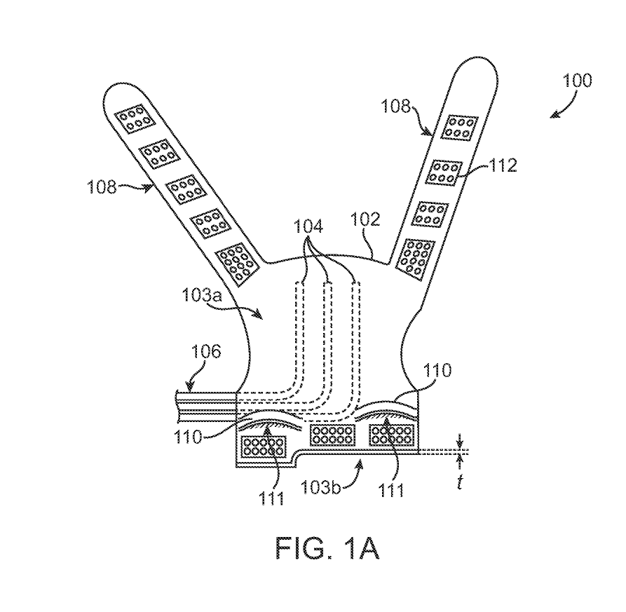

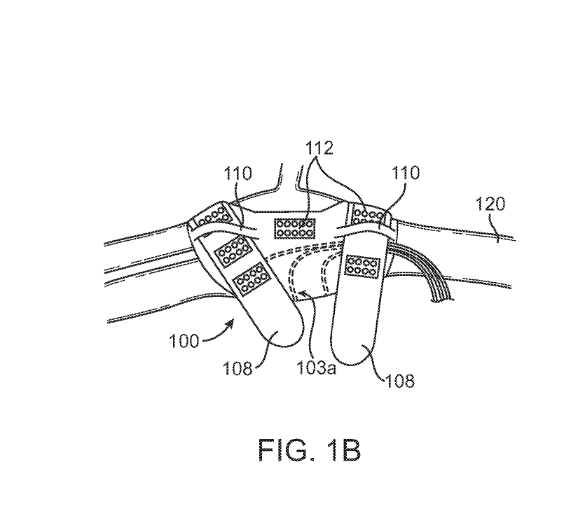

FIG. 1A is a perspective view diagram illustrating an implantable electrode assembly 100 according to one aspect of the invention. Electrode assembly 100 includes a base 102 that is preferably made from a thin, flexible elastomeric material or materials. In one example embodiment. base 102 is formed from silicone. Other suitable (e.g. bio-compatible) elastomers can be used. Base 102 can be molded, die-cut or fabricated by any suitable process known in the art.

In a related embodiment, base 102 is a multi-layer structure made from a combination of layers of different materials. In this type of embodiment, at least the outer-most layers (and, preferably, all materials) are made of bio-compatible material. In one embodiment, the base includes a resilient material that tends to favor a particular shape or structure, such as a cuff. This type of embodiment is described in greater detail in the co-pending and incorporated-by-reference related U.S. patent application cross-referenced above. In related embodiments, different positions of the base 102 can be made of different materials.

Base 102 has a pair of opposing major surfaces 103a (top surface) and 103b (bottom surface) that are separated by a base thickness t. On bottom surface 103b are situated a set of electrodes 104 for applying electrotherapy or electrostimulation to target tissue or for sensing electrical activity. Electrodes 104 are made from a conductive material that is preferably bio-compatible and are preferably designed to be flexible and elastic so that they can bend and stretch with the base 102. The flexible and preferably elastic properties of base 102 and electrodes 104 permit electrode assembly 100 to be conformed to a surface of the target tissue. In particular, as described in greater detail below, electrode assembly 100 is adapted to be wrapped around an elongate biological structure, such as, for example, a blood vessel, a nerve, a bone, or other such structure. Each electrode 104 is connected to, or integrally formed with, a corresponding lead wire 106 that connects the electrode to an output or input node of a signal generator or sensing circuit, respectively.

Electrode assembly 100 includes elongate straps, fingers, protrusions, or extensions (collectively referred to as straps) 108 that are used to secure electrode assembly 100 to the biological structure by wrapping around the outer surface of the biological structure. As is described in greater detail below, straps 108 can be integrally formed with base 102, or can be suitably attached to base 102. In one embodiment, as depicted in FIG. 1A, electrode assembly 100 also includes buckle features 110. Buckles 110 are designed to engage with straps 108 and to hold straps 108 in place once they are engaged with the corresponding buckles 110.

Together, each strap 108 and buckle 110 make up a belt mechanism for securing electrode assembly 100 to the biological structure. In one embodiment, as depicted in FIG. 1A, each strap 108 and buckle 110 achieve the retention effect by utilizing friction. For example, as depicted in FIG. 1A, buckle 110 is formed from an arching piece of base material situated over top surface 103a. Between the top surface 103a and the interior surface of the arch is a passage 111 through which strap 108 can be threaded. Once strap 108 has been threaded through passage 111, friction and adhesion forces between the outer surfaces of strap 108 and the interior surfaces of passage 111 will tend to bind the portion of strap 108 which is in intimate contact with passage 111 to buckle 110.

Optionally, electrode assembly includes a plurality of suture sites 112, each of which provides a reinforced portion of material that can be surgically sutured to the biological structure. For example, the suture sites 112 can be used to further secure electrode assembly 100 to arterial wall 120. In one embodiment, each suture site 112 is made from a mesh of flexible but non-elastic material that is encapsulated within the elastomeric base material. The mesh material is made from fibers having a tensile strength that is greater than the tensile strength of the elastomeric encapsulating material. Suture sites 112 prevent suture threads from tearing through the relatively softer elastomeric material, and permit securing electrode assembly to the implantation site with greater force.

FIG. 1B illustrates electrode assembly 100 affixed around an arterial wall 120, namely, at the carotid bifurcation. The implantation site is along a perimeter of the carotid bifurcation. The perimeter has a length around which electrode assembly 100 is wrapped. Upper surface 103a can be seen, while bottom surface 103b, upon which the electrodes are situated, is in intimate contact with the exterior surface of arterial wall 120. As illustrated, base 102 does not have a wrapping length that is sufficient to fully wrap around a circumferential length of the perimeter of the carotid bifurcation. However, base 102, in combination with each one of straps 108, achieves corresponding lengths, each of which is sufficient to fully wrap around the perimeter of the implantation site. Preferably, the combination lengths are substantially longer than the circumferential lengths of the perimeter, as illustrated, for facilitating threading straps 108 through buckles 110, leaving some strap length to spare.

Straps 108 are threaded through buckles 110 and pulled tight. The straps are secured with friction and/or adhesion forces, which must be overcome to loosen electrode assembly 100 from the artery 120. In an embodiment, surface features such as bumps, indentations, microstructures, or nanostructures are provided on one or more of the surface of the straps 108 and/or buckles 110 to enhance or reduce the friction and/or adhesion forces.

Preferably, the friction or adhesion forces are sufficient to maintain the position of electrode assembly 100 securely around the biological structure. When the belt mechanism is tightened, the straps 108 are stretched, and exert an elastic force that tends to return straps 108 to their original shape. In one embodiment, the belt mechanism is configured such that the adhesion or friction binding forces are balanced against a certain amount of elastic force established when the belt mechanism is tightened. In this embodiment, an elastic force having a magnitude that exceeds the binding force in the buckle (i.e. over-tightening) causes straps 108 to loosen until the binding force prevails. A preferred configuration of this type will maintain the secure attachment to the biological structure while preventing over-tightening of the belt mechanism. This characteristic is desirable when implanting electrode assembly 100 around a hollow or deformable biological structure such as a blood vessel.

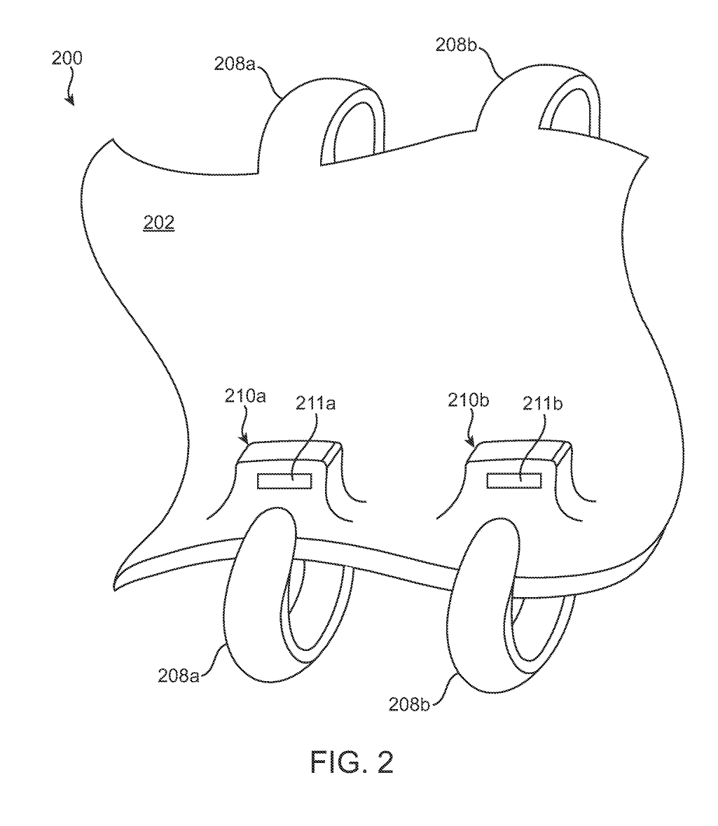

FIG. 2 is a perspective view diagram illustrating a portion 200 of an implantable electrode assembly according to one embodiment. Electrode assembly portion 200 has a base material 202, which has a pair of extensions comprising straps 208a and 208b. In this embodiment, straps 208a and 208b (collectively referred to as straps 208) are integrally formed with base 202. Base 202 also has buckle structures 210a and 210b (collectively referred to as buckles 210) protruding from the upper surface. In this embodiment, buckles 210 are also integrally formed with base 202. Buckles 210a and 210b each include, respectively, passages 211a and 211b (collectively referred to as passages 211). Passages 211 are situated so that they are generally parallel with the top surface of base 202 as illustrated. In operation, straps 208 are threaded through buckles 210 via passages 211, where they are retained.

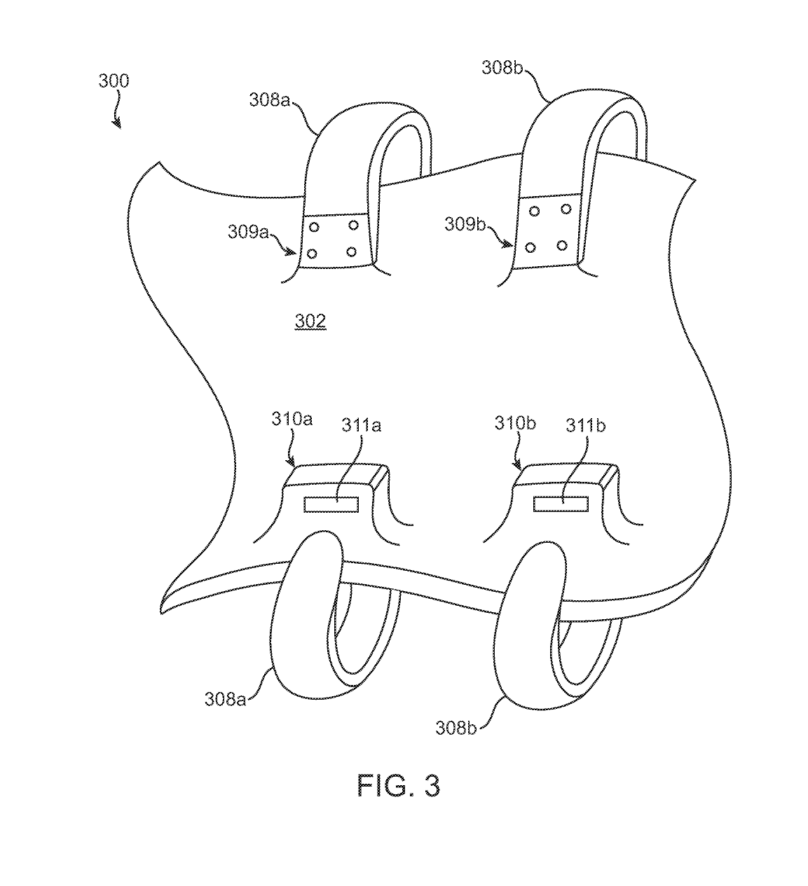

FIG. 3 is a diagram illustrating a portion 300 of an implantable electrode assembly according to another embodiment. Implantable electrode assembly portion 300 includes a base 302, on which are formed buckles 310a and 310b, having passages 311a and 311b, respectively, which are similar to the analogous elements described above with reference to FIG. 2. In this embodiment, straps 308a and 308b, however, are not integrally formed with the base 302. Rather, straps 308a and 308b are attached with, or coupled to, base 302 via fastener sets 309a and 309b, respectively. In a related embodiment (not shown), buckles 310a and 310b can be fastened to base 302 similarly to the way in which straps 308a and 308b are fastened thereto.

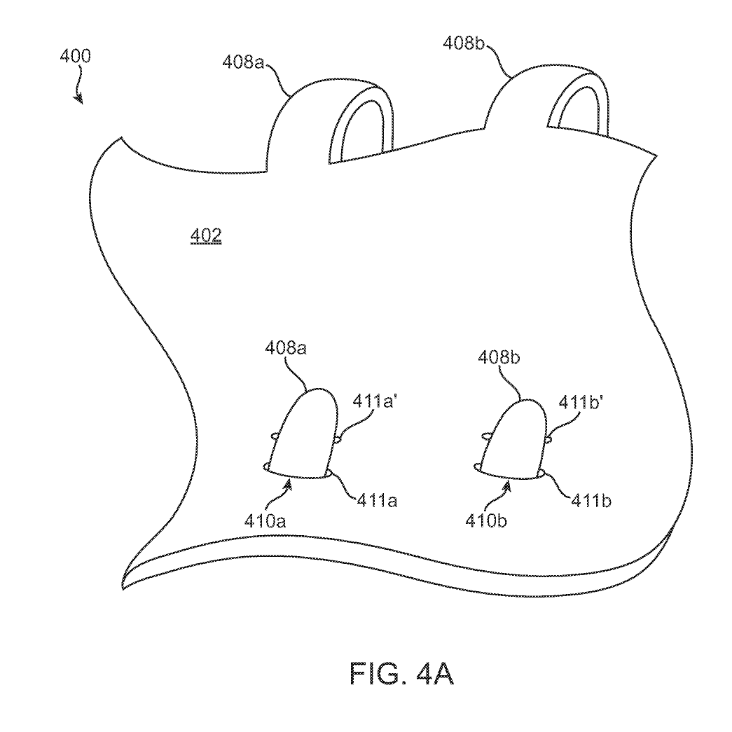

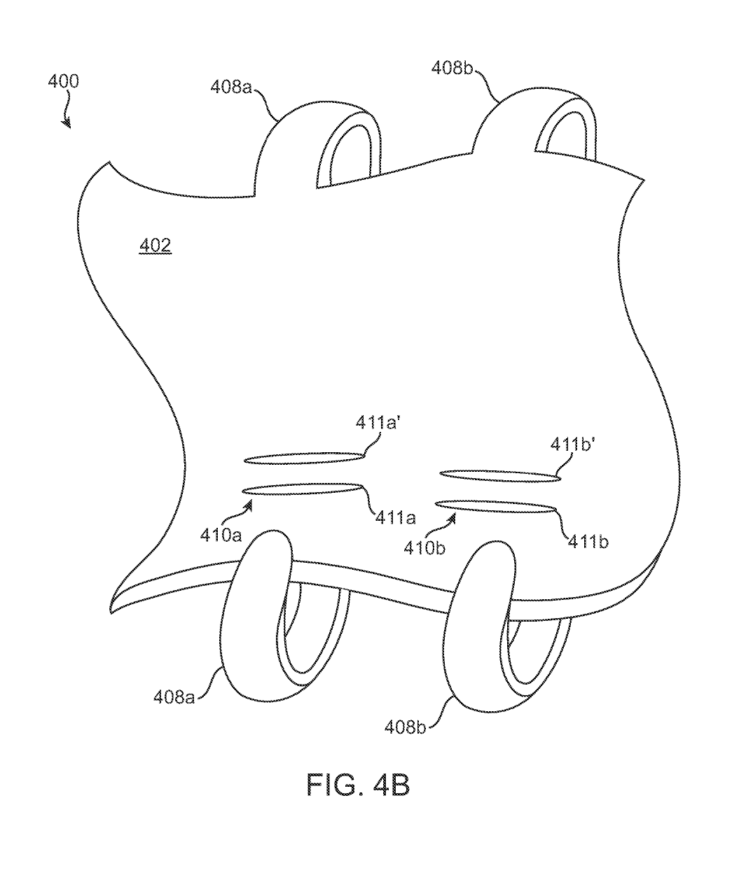

According to another embodiment, as illustrated in FIG. 4A, buckles 410a and 410b are each formed in base 402 of electrode assembly portion 400 as slits indicated at 411a, 411a′, 411b, and 411b′ (collectively referred to as slits 411). Each of slits 411 is a passage extending through the thickness of base 402 from the top surface to the bottom surface. Slits 411a and 411a′, together with the material of base 402 in which these slits are defined, constitute buckles 410a. Likewise, Slits 411b and 411b′, together with the material of base 402 in which these slits are defined, constitute buckles 410b.

In one type of embodiment, only slits 411a and 411b are present. Straps 408a and 408b (collectively, straps 408) can each be threaded through their respective slits 411 a and 411b as depicted. The interior walls of slits 411 retain straps 408 by friction or adhesion. Preferably, in the embodiment depicted in FIG. 4A, secondary slits 411a′ and 411b′ are present through which straps 408 can be further threaded. The additional looping back and threading through secondary slits 411a′ and 411b′ can substantially increase the friction and adhesive retention forces. Moreover, when straps 408 are threaded as depicted in FIG. 4A, the ends of straps 408 end up beneath the bottom surface of base 402, where they are held between base 402 and the biological structure (not shown) to which electrode assembly portion 400 is secured. This arrangement provides additional retention force.

In a related embodiment, as depicted in FIG. 4B, straps 408a and 408b are threaded first into slits 411a and 411b, respectively, towards the biological structure, and then out of slits 411a′ and 411b′. This type of securing arrangement for buckles 410a and 410b facilitates tightening straps 408 since their ends are ultimately protruding out of slits 411a′ and 411b′ on the top surface of base 402 and are therefore more easily accessible.

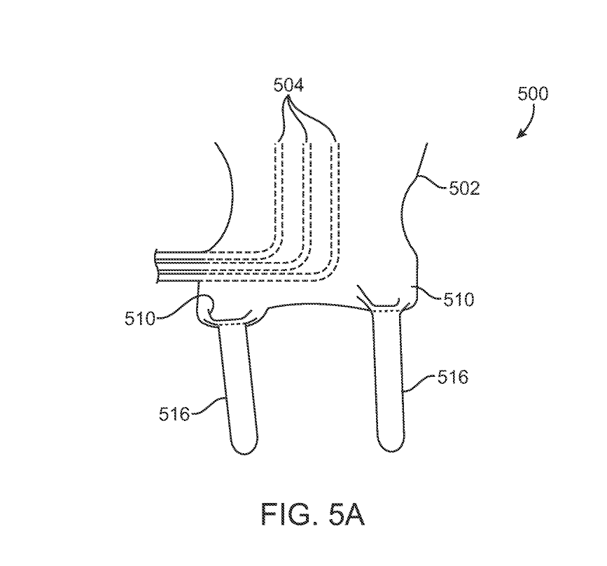

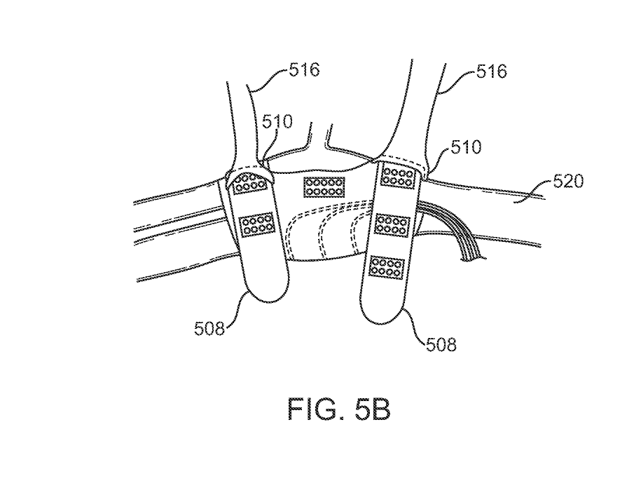

FIGS. 5A, 5B, and 6-8 all illustrate related embodiments having a buckle with a release tab for facilitating threading the strap through the passage of the buckle, and for releasing the retention force for loosening the electrode assembly from the biological structure to which it was attached. Referring to FIG. 5A, a portion 500 of an implantable electrode assembly is depicted. Electrode assembly portion 500 includes a base 502, on which a set of electrodes 504 is attached. Buckles 510 of the type described above with reference to FIGS. 1A and 1B are either integrally formed with base 502, or attached thereto. Each buckle 510 has a release tab 516 that provides a convenient grip for manipulating the arch portion of buckle 510. In one embodiment, release tab 516 is an elongate portion of material having a belt-like shape. In one type of embodiment, release tab 516 is integrally formed with a portion of buckle 510. In another embodiment, release tab is attached to the portion of buckle 510.

FIG. 5B illustrates the electrode assembly of FIG. 5A affixed around blood vessel 520. Belts 508 are threaded through buckles 510 and pulled tight. Release tabs 516 can be pulled to reduce the binding force between the interior surface of buckles 510 and the exterior surface of belts 508, thereby permitting the attachment of electrode assembly 500 to be loosened and re-adjusted.

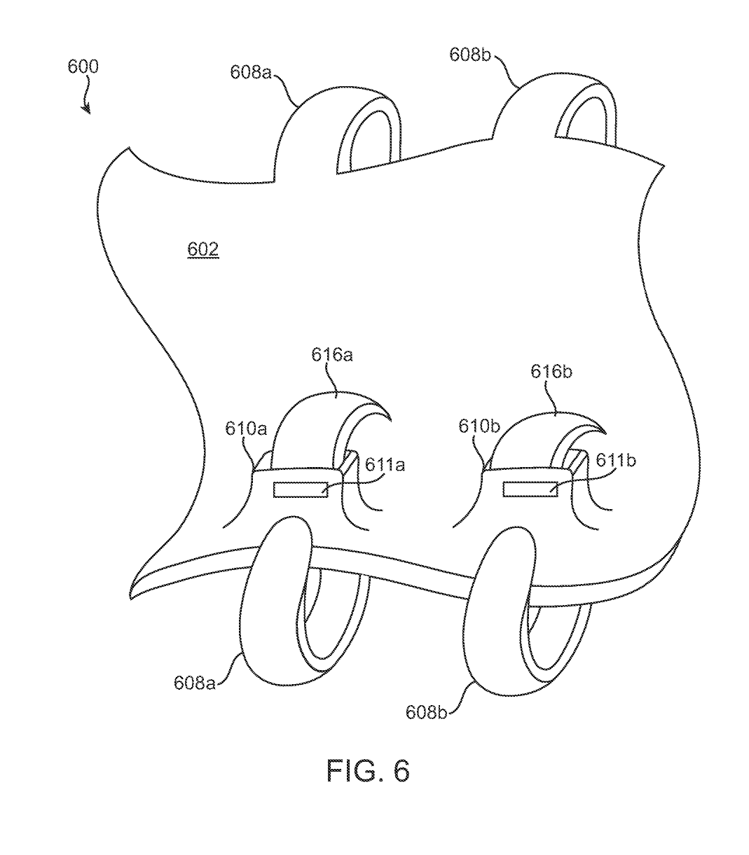

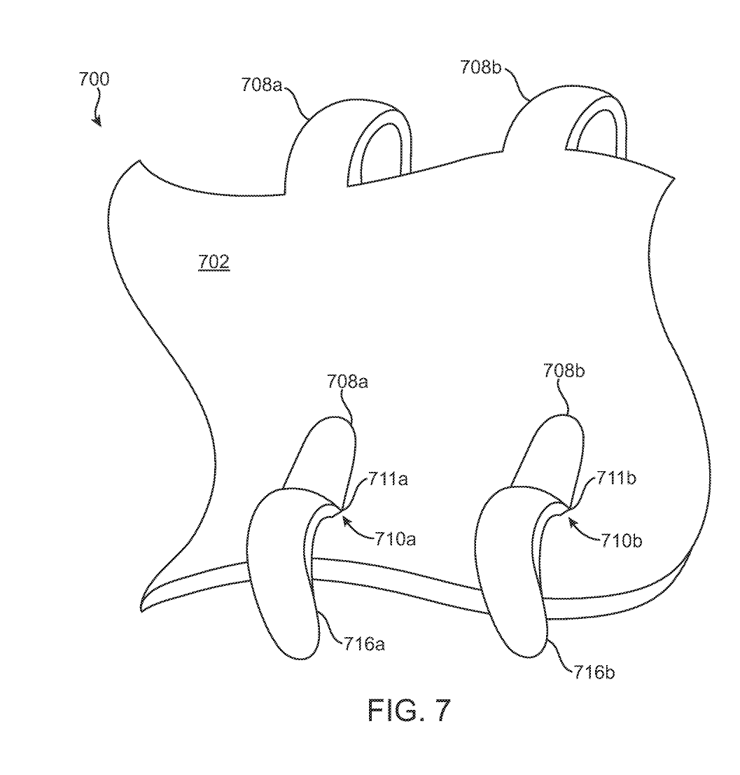

FIG. 6 illustrates a portion 600 of an electrode assembly according to one embodiment. Electrode assembly portion 600 includes a base 602, with which are integrally-formed belts 608a and 608b, and buckles 610a and 610b having passages 611a and 611b, respectively. Each buckle 610a and 610b also includes a release tab 616a and 616b as depicted. An upward pull on release tab 616a, for example, stretches the material from which the corresponding buckle 610a is formed, and expands the cross-sectional size of the corresponding passage 611a. As discussed above with reference to FIG. 5A, expanding the size of passage 611a permits belt 608a to be more easily threaded through the corresponding buckle 610a. Likewise, expanding the size of passage 611a permits the corresponding buckle 610a to be loosened around an inserted strap 608a for adjustment and removal.

FIG. 7 illustrates an example electrode assembly portion 700 having base 702, straps 708a and 708b, and a pair of slit-type buckles 710a and 710b formed in base 702. The slits define a pair of passages 711a and 711b through base 702, through which a corresponding pair of belts 708a and 708b can be threaded as shown. A pair of release tabs 716a and 716b are secured to base 702 near the corresponding slits. Tabs 716a and 716b can be pulled to loosen the slits to facilitate insertion, positional adjustment, and removal of belts 708.

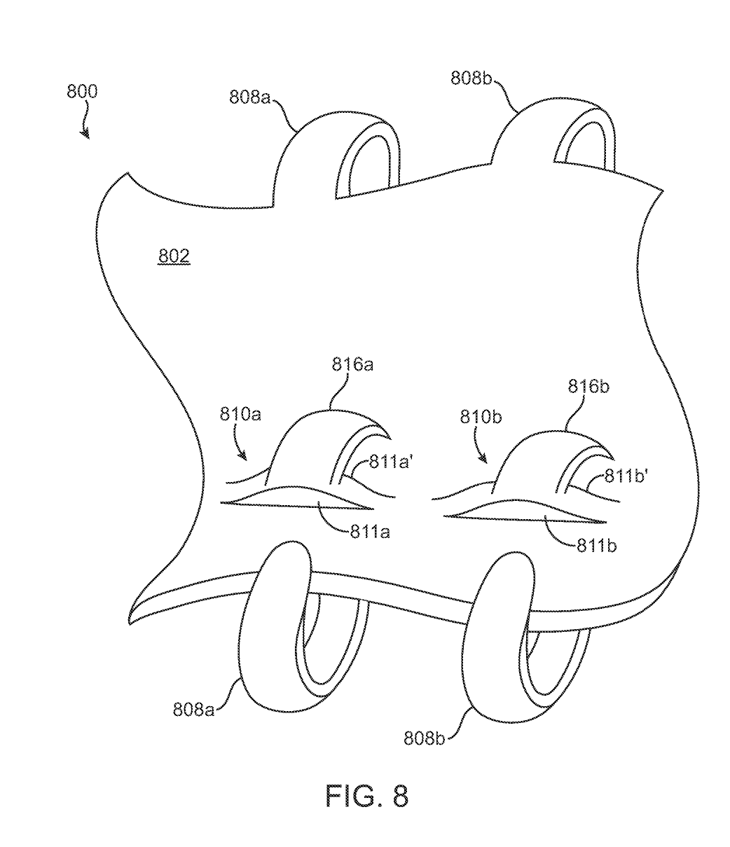

FIG. 8 illustrates a related embodiment in which an electrode assembly portion 800 includes a dual slit-type buckle arrangement such as the one described above with reference to FIGS. 4A and 4B. Buckles 810a and 810b are formed integrally with base 802 by slits 811a and 811a′, and 811b and 811b′. Each buckle 810a, 810b also includes a release tab 816a, 816b that, when pulled upwards, expands the size of passages 811a-811a′ and 811b-811b′, thereby permitting straps 808a and 808b to be more easily inserted therethrough, removed, and adjusted as desired.

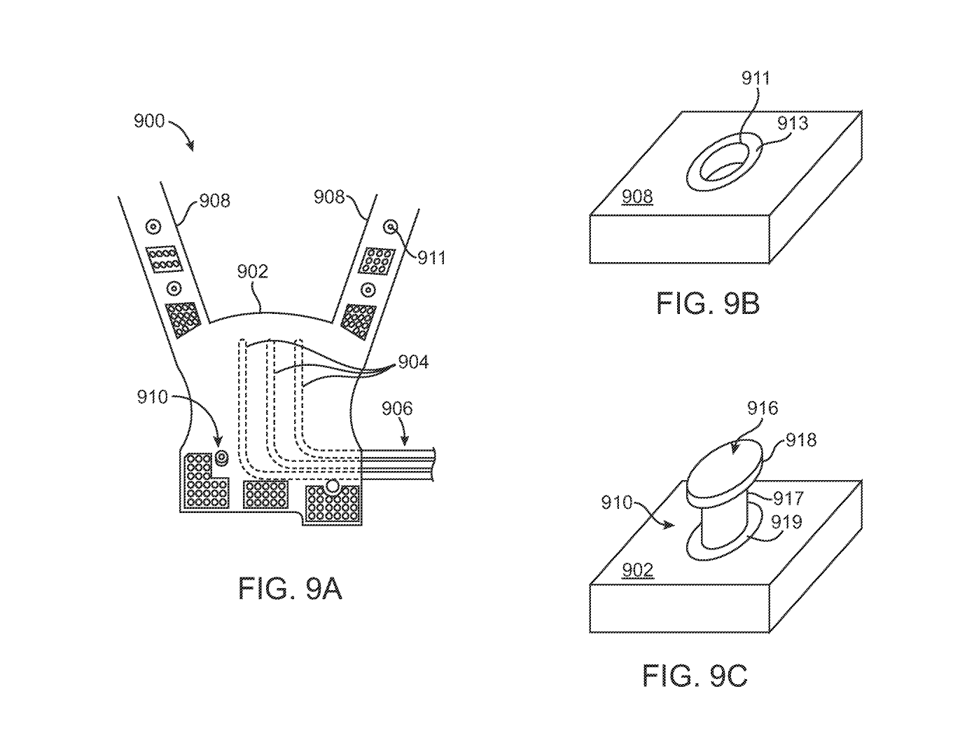

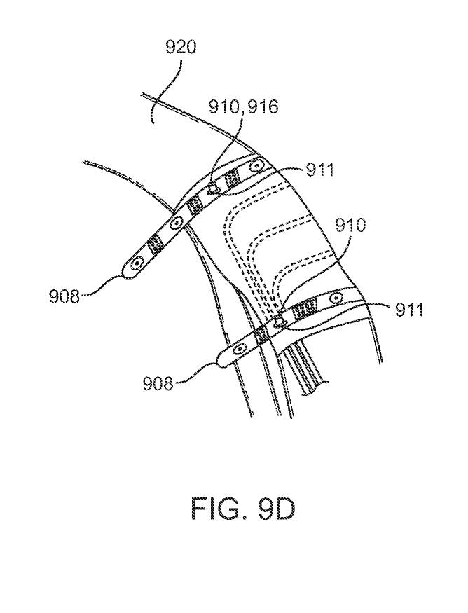

FIGS. 9A-9D illustrate an example electrode assembly 900 having a button-type belt mechanism. Electrode assembly 900 includes a base 902, on which are positioned electrodes 904 having lead wires 906. Electrode assembly 900 also includes straps 908, which can be integrally formed with, or attached to, base 902. Each of straps 908 include a plurality of through holes 911 that are essentially passages through the strap from the top surface to the bottom surface. The buckle 910 includes a pin, a tab, or other protrusion that can engage with one or more through holes 911. In a related embodiment (not shown), a set of protrusions can be positioned along a length of the strap, while the mating through hole can be located at the buckle position 910.

An example of a through hole 911 is depicted in FIG. 9B. Optionally, through hole 911 is reinforced with a reinforcing ring 913 of resilient material that requires a greater force to be deformed as compared with the material of strap 908. Reinforcing ring 913 can be embedded in strap 908 as depicted in FIG. 9B. Through hole 911 and reinforcing ring can have other cross-sectional shapes, such as rectangular or hexagonal cross-sections. In one embodiment, through hole 911 is in the form of a slit that can be elastically expanded to widen the passage through strap 908.

Referring again to FIG. 9A, the plurality of through holes positioned along the length of each strap 908 are preferably positioned at a spacing interval that enables the surgeon to secure the example electrode assembly 900 around the biological structure with a suitable tension. Thus, preferably, the granularity of incremental tensions should be sufficiently fine to permit selecting a tension point within a suitable range.

FIG. 9C illustrates an example of a protrusion 916 of buckle 910. As depicted, protrusion 916 includes a generally cylindrical stem portion 918 that has a lower portion embedded in base material 902 and an upper protruding portion 917. In other embodiments, stem portion 918 can have a non-cylindrical cross-section, such as a rectangular or hexagonal cross-section. The lower portion of stem portion 917 can have a base or some radial feature (neither shown) for facilitating retention of stem portion 917 in the base material 902.

Preferably, the upper protruding portion of stem 917 is taller than the thickness of strap 908. Protrusion 916 also preferably includes a head portion 918 that has a diameter greater than the diameter of the upper protruding portion stem portion 917. To further facilitate secure retention of strap 908, head portion 918 is has a cross-sectional area (e.g. diameter) that is slightly larger than the corresponding cross-sectional area (e.g. diameter) of through holes 911. Optionally, a reinforcing portion, such as reinforcing ring 919 made of resilient material is embedded in base material 902 to help retain protrusion 916.

FIG. 9D is a perspective view diagram illustrating example electrode assembly 900 secured to a carotid bifurcation 920. Buckles 910, having protrusions 916, are mutually engaged with through holes 911 as indicated. Straps 908 can be manipulated to disengage and re-engage the belt mechanisms as part of finding an optimal position for securing electrode assembly 900 to the artery.

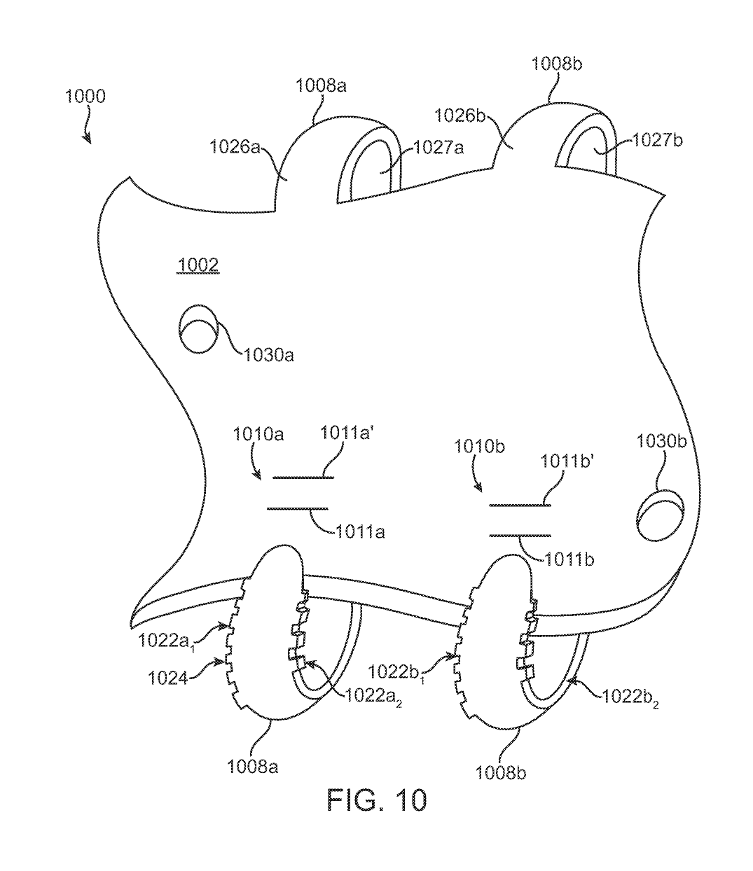

FIG. 10 is a diagram illustrating a portion 1000 of an implantable electrode assembly having a base 1002, and slot-type buckles 1010a and 1010b having slots 1011a, 1011a′, 1011b, and 1011b′ (collectively, slots 1011) that are similar to the slots of slot-type buckles 410a and 410b described above with reference to FIGS. 4A and 4B. Straps 1008a and 1008b respectively include surfaces 1022a₁ and 1022a₂, and 1022b₁ and 1022b₂ (collectively, surfaces 1022) as shown. Each of these surfaces includes a set of protrusions 1024 that operate to increase the friction/adhesive binding force between the interior surfaces of slots 1011 and surfaces 1022.

In a related embodiment, protrusions such as protrusions 1024 can be present on top surfaces 1026a and 1026b and/or bottom surfaces 1027a and 1027b of straps 1008a and 1008, respectively. Protrusions 1024, as depicted in FIG. 10, are rectangular notches. However, protrusions 1024 can take any suitable pattern, including, but not limited to, notches, serrations, undulations, teeth, steps, and surface texture.

Example electrode assembly portion 1000 further includes a pair of apertures 1030a and 1030b, which can be used by the surgeon to mark an optimal position on the implantation site. This can be useful for re-positioning the electrode assembly in a particular alignment that was found to provide an optimal administration of electrotherapy, for example.

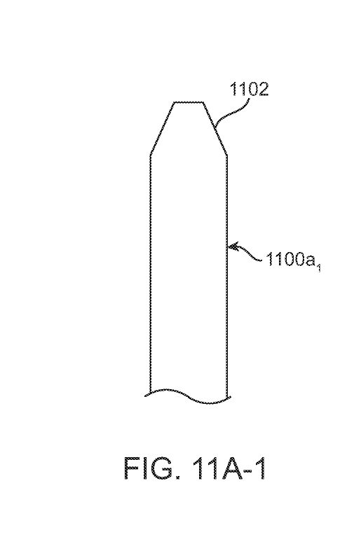

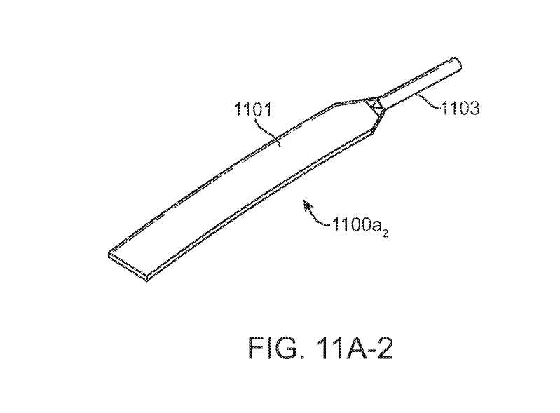

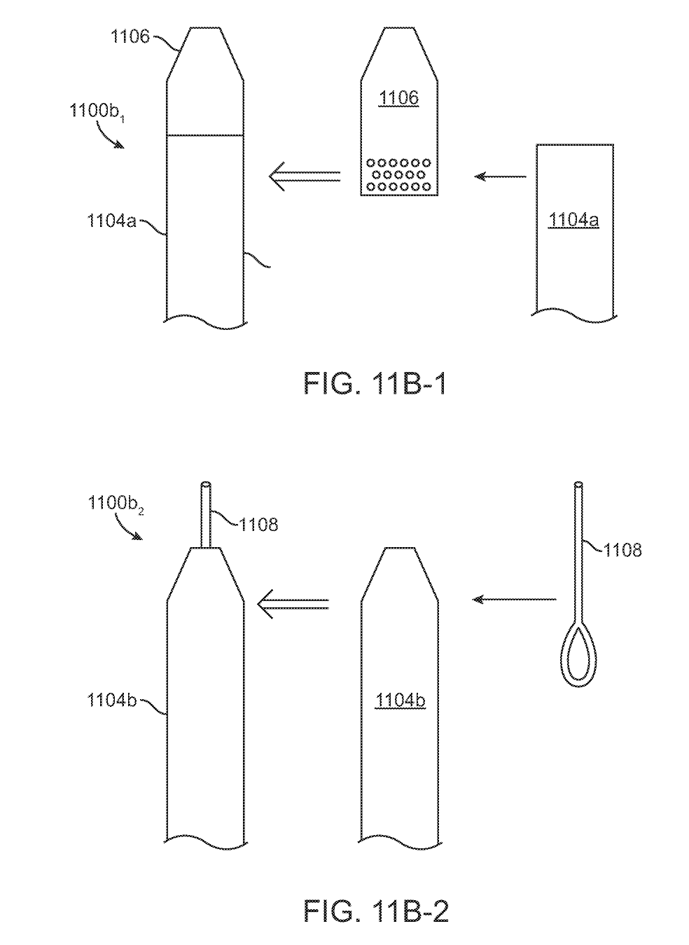

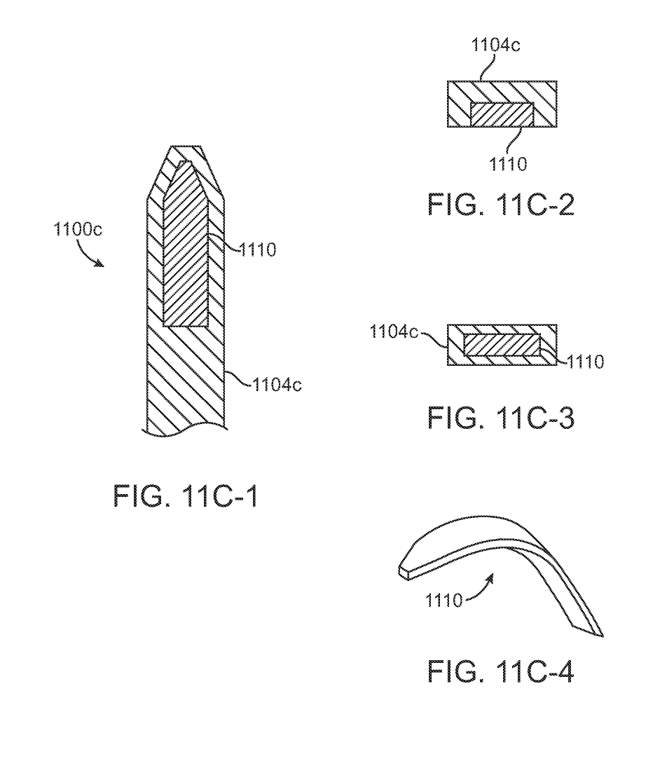

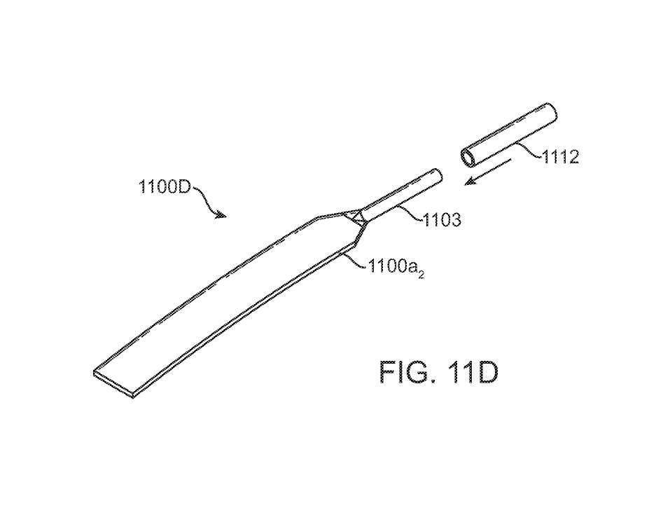

According to one type of embodiment of the belt mechanism, each of the strap(s) includes one or more end features that facilitate threading the strap through the buckle. FIGS. 11A through 11C-4 illustrate several example embodiments of such features. FIG. 11A-1 depicts one-part construction of strap portion 1100a₁, which has a tapered tip 1102. Tapered tip 1102 can take any suitable form, such as a triangular tip, or a rounded tip.

FIG. 11A-2 illustrates strap portion 1100a₂, which is another example of a one part construction. Strap portion 1100a₂ includes a main portion 1101 and an integral leader portion 1103. In one embodiment, strap portion 1100a₂ is formed by molding leader portion 1103 and main portion 1101 together. Leader portion 1103 can be formed with a straight shape (as depicted), or with a curved shape. In one embodiment, leader portion 1103 is dimensioned to be more resilient than main portion 1101, such as, for example, by having a greater thickness than that of main portion 1101.

FIG. 11B-1 illustrates am example strap portion 1100b₁ that includes a two-part construction. Strap portion 1100b₁ has a main portion 1104a that is integral with, or attached to, the base of the electrode assembly (not shown), and also has a tip portion 1106 that is made from a different material than main portion 1104a. In one such embodiment, tip portion 1106 is made from a relatively more resilient material, such as, for example, nylon, or a more resilient elastomer. Tip portion 1106 can be attached to, or formed with main portion 1104a. In one embodiment, as illustrated, tip portion can be co-molded with main portion 104. Tip portion 1106 can be pre-formed with retention features, such as holes or surface features, to facilitate attachment to, or partial encapsulation by main portion 1104a.

FIG. 11B-2 illustrates another example embodiment of a two-part construction. Strap portion 1100b₂ includes main portion 1104b and a leader portion 1108 formed from a more resilient material. When assembled or fabricated, leader portion 1108 protrudes from the end of main portion 1104b. As depicted in this example, leader portion 1108 can be pre-formed and partially encapsulated in main portion 1104b.

FIGS. 11C-1 through 11C-4 illustrate various embodiments of a two-part strap end that has a reinforcing portion for facilitating end rigidity. In FIG. 11C-1, a cross-sectional view is shown depicting a strap portion 1100c having a main portion 1104c and a reinforcing portion 1110. Reinforcing portion 1110 can be made from a material that is more resilient than main portion 1104c. As illustrated in FIG. 11C-2, reinforcing portion 1110 can be partially encapsulated by main portion 1104c. Alternatively, as illustrated in FIG. 11C-3, reinforcing portion 1110 can be entirely encapsulated in main portion 1104c.

FIG. 11C-4 illustrates one type of embodiment in which reinforcing portion 1110 is pre-formed with a curved shape that causes strap portion 1100c to retain a correspondingly curved shape that further facilitates threading strap portion 1100c through the buckle of the electrode assembly. Referring again to FIG. 11B-1, tip portion 1106 can be similarly curved in one embodiment.

FIG. 11D illustrates another embodiment of a strap that employs a reinforcing portion. Strap portion 1100D is a multi-part design that includes strap portion 1100a₂ with leader portion 1103 described above with reference to FIG. 11A-1. Strap portion 1100D further includes reinforcing portion 1112 that is made from a more resilient material than that of strap portion 1100a₂. Reinforcing portion 1112 is in the shape of a sleeve adapted to fit over leader portion 1103. Persons of ordinary skill in the relevant arts will appreciate that reinforcing portion 1112 can be affixed to strap portion 1100a₂ by a variety of suitable mechanisms. For example, reinforcing portion 1112 can be affixed to leader portion 1103 with an adhesive. Reinforcing portion 1112 can also be compression fitted over the leader portion 1103 by being undersized so as to create a friction fit. Other mechanisms include deforming reinforcing portion 1112 over leader portion 1103 such as by crimping. Another possible approach includes shrinking the reinforcing portion 1112 material onto leader portion 1103 using known methods, such as, for example, via thermal, chemical or luminescent exposure.

Various modifications to the invention may be apparent to one of skill in the art upon reading this disclosure. For example, persons of ordinary skill in the relevant art will recognize that the various features described for the different embodiments of the invention can be suitably combined, un-combined, and re-combined with other features, alone, or in different combinations, within the spirit of the invention. Likewise, the various features described above should all be regarded as example embodiments, rather than limitations to the scope or spirit of the invention. Therefore, the above is not contemplated to limit the scope of the present invention, which is limited only by the appended claims and their equivalents.

What is claimed is:

1. An electrode assembly for implantation in a wrapped configuration around an outer surface of a blood vessel structure at an implantation site along a perimeter of the blood vessel structure, the electrode assembly comprising:
a generally flexible base having a pair of opposing major surfaces, wherein the base conforms to the outer surface of the blood vessel structure and has a wrapping length that is at least a portion of a circumferential length of the perimeter of the blood vessel structure when the electrode assembly is implanted at the implantation site;
a set of electrodes secured over at least one of the major surfaces of the base;
at least one belt mechanism that secures the electrode assembly at the implantation site, wherein each of the at least one belt mechanism includes a strap and a buckle, wherein the buckle retains a portion of the strap when the strap is operably engaged with the buckle; and
wherein a combination of the base and at least one of the at least one belt mechanism has a combined wrapping length that is at least the circumferential length of the perimeter.

2. The electrode assembly of claim 1, wherein the base has a wrapping length that is less than the circumferential length of the perimeter.

3. The electrode assembly of claim 1, wherein the base material is an elastomer.

4. The electrode assembly of claim 1, wherein the electrode assembly comprises a plurality of belt mechanisms.

5. The electrode assembly of claim 1, wherein the electrode assembly further comprises a plurality of reinforced suture sites.

6. The electrode assembly of claim 1, wherein at least one of the buckle and the strap is integrally formed with the base.

7. The electrode assembly of claim 1, wherein at least one of the buckle and the strap is attached to the base.

8. The electrode assembly of claim 1, wherein the buckle is formed in the base and defines at least one passage through the pair of opposing major surfaces.

9. The electrode assembly of claim 8, wherein the at least one passage through the pair of opposing major surfaces is at least one slit.

10. The electrode assembly of claim 1, wherein the buckle is situated over a corresponding one of the major surfaces such that the buckle defines a passage situated over the corresponding one of the major surfaces.

11. The electrode assembly of claim 10, wherein the buckle includes an arch situated over the corresponding one of the major surfaces.

12. The electrode assembly of claim 1, wherein the buckle defines a passage and includes a release tab, wherein the release tab facilitates widening the passage.

13. The electrode assembly of claim 1, wherein the at least one belt mechanism includes a mating set of a through hole and a protrusion that engages with the through hole.

14. The electrode assembly of claim 13, wherein the mating set is configured according to at least one configuration selected from the group consisting of:
(a) the through hole defined in the strap, and the protrusion being a part of the buckle;
(b) the through hole defined in the buckle, and the protrusion being a part of the strap;
(c) the protrusion situated generally perpendicularly to the pair of opposing major surfaces;
(d) the protrusion being reinforced with a resilient material; and
(e) the through hole being reinforced with a resilient material.

15. The electrode assembly of claim 1, wherein the strap includes a first surface, at least a portion which includes a set of surface features that increase retention strength between the buckle and the strap.

16. The electrode assembly of claim 15, wherein the set of surface features includes at least one protrusion pattern selected from the group consisting of: notches, serrations, undulations, bumps, teeth, steps, and surface texture, and surface coating.

17. The electrode assembly of claim 1, wherein the strap includes at least one end feature that facilitates insertion of the strap for engagement with the buckle.

18. The electrode assembly of claim 17, wherein the at least one end feature is selected from the group consisting of:
(a) a curved tip;
(b) a tip comprising a more rigid material than that of the base;
(c) a reinforcing portion;
(d) a leader portion; and
(e) a multipart tip comprising at least two different materials.

19. The electrode assembly of claim 1, wherein the base includes a set of at least one aperture defined therein for facilitating making a mark on the blood vessel structure during implantation of the electrode assembly.

20. An electrode assembly for implantation around an elongate biological structure, the electrode assembly comprising:
means for retaining a set of electrodes;
belt means for securing the electrode assembly around an outer surface of the elongate biological structure, the belt mechanism including:
strap means for permitting the electrode assembly to wrap around the outer surface of the elongate biological structure; and
buckle means for retaining a portion of the strap means.

21. An electrode assembly for implantation in a wrapped configuration around an outer surface of an elongate biological structure at an implantation site along a perimeter of the elongate biological structure, the electrode assembly comprising:
a generally flexible base having a pair of opposing major surfaces, wherein the base conforms to the outer surface of the elongate biological structure and has a wrapping length that is at least a portion of a circumferential length of the perimeter of the elongate biological structure when the electrode assembly is implanted at the implantation site;
a set of electrodes secured over at least one of the major surfaces of the base;
at least one belt mechanism that secures the electrode assembly at the implantation site, wherein each of the at least one belt mechanism includes a strap and a buckle, wherein the buckle retains a portion of the strap when the strap is operably engaged with the buckle; and
wherein a combination of the base and at least one of the at least one belt mechanism has a combined wrapping length that is at least the circumferential length of the perimeter.

22. The electrode assembly of claim 21, wherein the base has a wrapping length that is less than the circumferential length of the perimeter.

23. The electrode assembly of claim 21, wherein the electrode assembly comprises a plurality of belt mechanisms.

24. The electrode assembly of claim 21, wherein the buckle is formed in the base and defines at least one passage through the pair of opposing major surfaces.

25. The electrode assembly of claim 21, wherein the buckle is situated over a corresponding one of the major surfaces such that the buckle defines a passage situated over the corresponding one of the major surfaces.

26. The electrode assembly of claim 25, wherein the buckle includes an arch situated over the corresponding one of the major surfaces.

27. The electrode assembly of claim 21, wherein the buckle defines a passage and includes a release tab, wherein the release tab facilitates widening the passage.

28. The electrode assembly of claim 21, wherein the at least one belt mechanism includes a mating set of a through hole and a protrusion that engages with the through hole.

29. The electrode assembly of claim 21, wherein the strap includes a first surface, at least a portion which includes a set of surface features that increase retention strength between the buckle and the strap.

30. The electrode assembly of claim 21, wherein the strap includes at least one end feature that facilitates insertion of the strap for engagement with the buckle.

31. The electrode assembly of claim 21, wherein the base includes a set of at least one aperture defined therein for facilitating making a mark on the elongate biological structure during implantation of the electrode assembly.

32. A method of implanting an electrode assembly around an elongate biological structure, the method comprising:
providing at least one belt set including a strap and a buckle as part of the electrode assembly;
wrapping a portion of an outer surface the elongate biological structure with the electrode assembly including with the strap;
engaging the strap with the buckle such that the buckle retains an engaged portion of the strap.

33. The method of claim 32, wherein the step of engaging includes threading the at least one strap through a passage defined by the at least one buckle such that the strap and the buckle bind with one another by friction or adhesive retention force.

34. The method of claim 33, wherein the step of engaging includes pulling on a release tab of the buckle to elastically expand the passage.

35. The method of claim 32, wherein the step of engaging includes inserting a protruding buckle portion through one of a series of passages defined through the at least one strap.

36. The method of claim 32, wherein the step of engaging includes:
over-tightening the strap by pulling the strap through the buckle;
after the over-tightening, releasing the strap such that the strap elastically returns towards its un-stretched position at a final tension; and
maintaining the final tension.

37. The method of claim 32, further comprising marking a point on the outer surface of the elongate biological structure through an aperture defined in the base.
